(19) United States
(12) Patent Application Publication
(10) Pub. No.: US 2011/0284010 A1
(43) Pub. Date: Nov. 24, 2011

(54) CONDOM WITH COATING HAVING CAPSULES

(75) Inventors: Beng Sim Chuah, Petaling Jaya , SGR (MY); David M. Lucas, Selangor (MY); Dave Narasimhan, Flemington, NJ (US)

(73) Assignee: ANSELL HEALTHCARE PRODUCTS LLC, Red Bank, NJ (US)

(21) Appl. No.: 13/123,800

(22) PCT Filed: Jul. 31, 2009
(86) PCT No.: PCT/US2009/052330

§ 371 Date: Aug. 2, 2011

Related U.S. Application Data
(60) Provisional application No. 61/085,194, filed on Jul. 31, 2008.

(51) Int. Cl. A61F 6/04; B32B 38/08

(52) U.S. Cl. 128/844; 427/2.3; 156/297

(57) ABSTRACT

Provided are condoms, methods of making and treating sexual dysfunction with the same, the condoms having a multiplicity of microcapsules containing an ingredient therein, the microcapsules being disposed on one or more surfaces of the condom such that the microcapsules are substantially exposed. The microcapsules can be disposed on one or both surfaces. A barrier layer between the microcapsules and the condom can be provided. The barrier layer can comprise nitrile, polyurethane, polyacetal urea, or combinations thereof. Further embodiments of such condoms are provided where an ingredient without encapsulation is disposed on the elastomeric layer along with the microcapsules.

### TECHNICAL FIELD

Embodiments of the present invention relate to condoms having an encapsulated ingredient disposed on one or more surfaces, and methods of manufacturing thereof. Ingredients, for example, phosphodiesterase type 5 inhibitors, are suitable for treatment of sexual dysfunction.

### BACKGROUND

Condoms and other prophylactics and protective devices provide physical barriers against the transmission of bodily fluids or other fluids. Chemical barriers are also used alone or in conjunction with condoms to prevent such transmission. In some instances, the chemical barriers serve as a supplemental form of protection in the event the physical barrier is breached. Active ingredients can be delivered via condoms and other prophylactics and protective devices to treat, for example, sexual dysfunction in men and women. Topical application of therapeutic agents such as vasodilators is one option. As provided in U.S. Pat. Nos. 4,829,991 (Boeck) and 6,840,244 (Kemp), condoms can be used to deliver therapeutic agents such as vasodilators and erectogenic compounds. Delivery of such therapeutic agents to human tissue depends on, for example, stability of the agents in the formulation and the ability of the agent to penetrate the tissue. There can be certain limitations in the effectiveness of therapeutic agents based on their methods of delivery. For example, topical treatments to the penis alone are subject to being wiped off before being effective. In addition, topical treatments may be diluted in the presence of body fluids. Moreover, certain therapeutic agents may suffer from short shelf-lives.

Technologies for delivering therapeutic agents include the use of liposomes. For example, WO 2005/115337 (Polymun Scientific) provides liposomes for drug delivery, where a lipsosome contains a desired drug in aqueous phase in the interior of the liposome and one or more drugs attached to either or both sides of the liposomal membrane. In so doing, the drug used generally has a functional group that is reactive with a functional group of the lipid portion. U.S. Pat. No. 6,541,030 (Vaghefi) disclose water-soluble microcapsules and methods of encapsulation. U.S. Pat. No. 4,983,404 (Raman et al.) also discloses the use of encapsulation to control the release of compounds. U.S. Pat. No. 6,843,942 (Katinger et al.)

discloses apparatus and methods for producing lipid vesicles. U.S. Pat. No. 4,428,983 (Nehen et al.) and 4,379,071 (Schnoring et al.) disclose Process for the production of microcapsules.

There is a need to provide therapeutic agents for the purposes of treating sexual dysfunction in convenient ways that promote stability of the article containing the agent and ease of delivery of the agents. There is a need for a condom having one or more encapsulated ingredients disposed thereon.

### SUMMARY

Provided are condoms, methods of making and treating sexual dysfunction with the same, the condoms having a multiplicity of capsules, such as microcapsules and/or liposomes, containing an ingredient therein, the capsules being disposed on one or more surfaces of the condom such that the capsules are substantially exposed. In a detailed aspect, provided are condoms comprising an elastomeric layer and a multiplicity of microcapsules having a PDE-5 inhibitor contained therein, the capsules being disposed on a surface of the elastomeric layer such that the capsules are substantially exposed, wherein the PDE-5 inhibitor is selected from the group consisting of sildenafil, tadalafil, vardenafil, derivatives thereof, and pharmaceutically acceptable salts thereof.

While liposomes are capable of entraining both aqueous solutions as well as water-insoluble constituents, the lipid content of the liposome may interact with latex products such as natural rubber. Therefore, it may be desirable to avoid direct contact between the lipsosome microcapsules and the latex product by providing a barrier between then. One such barrier could also serve as an adhesive for attaching liposome microcapsules to the latex product. This adhesive may be a latex composition such as nitrile latex that resists oil and other lipids. Other ingredients suited as an adhesive include polyacetal urea produced by additive polymerization at low temperatures or any oil resistant polymer solution in a solvent that evaporates quickly.

Another aspect provides methods of manufacturing condoms, the methods comprising the steps of: providing a multiplicity of microcapsules having an ingredient therein; forming an elastomeric layer, wherein the elastomeric layer is selected from a group consisting of natural rubber latex, synthetic polyisoprene, guayule, polyurethane and copolymers or combinations thereof; and applying the multiplicity of microcapsules to a surface of the elastomeric layer, such that the microcapsules are substantially exposed. Often, the elastomeric layer is tacky upon formation by curing and/or drying. This tackiness is one way to secure the microcapsules to the elastomeric layer. Further methods can include providing a barrier between the microcapsules and the elastomeric layer. Also, the microcapsules can be adhesively bonded to the elastomeric layer.

In a further aspect, provided are methods for treating sexual dysfunction, the methods comprising providing a condom comprising an elastomeric layer and a multiplicity of microcapsules having a PDE-5 inhibitor contained therein, the microcapsules being disposed on a surface of the elastomeric layer such that the microcapsules are substantially exposed, wherein the PDE-5 inhibitor is selected from the group consisting of sildenafil, tadalafil, vardenafil, derivatives thereof, and pharmaceutically acceptable salts thereof. In a detailed embodiment, the treatment of sexual dysfunction is for males. Other embodiments provide that the treatment is for females. Still further, treatment can be for both males and females.

### DETAILED DESCRIPTION

It has been found that delivery of an active ingredient to treat sexual dysfunction can benefit from the use of closed capsules (microcapsules, liposomes, or the like) to contain the active ingredient. In this way, the active ingredient is kept contained within the capsules until it is needed. This is helpful for ingredients that may be less stable when exposed to air or have a short half-life. Moreover, the choice of microcapsule composition can enhance delivery of the active ingredients.

According to a first aspect, provided are condoms having a multiplicity of microcapsules containing an ingredient therein, the microcapsules being disposed on one or more surfaces of the condom such that the microcapsules are substantially exposed. The microcapsules can be adhesively bonded to the surface. In this way, should it be necessary, the microcapsules can be broken by friction. In other embodiments, liposomes, which contain the ingredient, are soluble in the skin layer and/or mucosal tissue. When liposomes are used for encapsulating the ingredient, controlled- or time-release of the ingredient can be achieved. In one or more embodiments, the capsules can be suspended in a lubricant on the surface of the condom.

In one or more embodiments, the ingredient in the microcapsules is selected from the group consisting of a microbicide, an anti-viral agent, a warming or cooling lubricant, a vasodilator, a spermicide, and compatible combinations thereof. Microbicides, anti-viral agents, and spermicides, once released, react with sperm or microorganisms upon contact on the skin or condom surface.

When the ingredient of the microcapsule is a vasodilator, the condom having microcapsules can be used to treat sexual dysfunction. In a specific embodiment, the ingredient in the microcapsules is an inhibitor of phosphodiesterase type 5, also referred to as a PDE-5 inhibitor. The PDE-5 inhibitor can be a compound selected from the group consisting of sildenafil, tadalafil, vardenafil, derivatives thereof, and pharmaceutically acceptable salts thereof. Another specific embodiment provides that the ingredient comprises a prostaglandin compound, such as prostaglandin E-1, which is known under the pharmaceutical name of alprostadil. Another suitable ingredient is testosterone.

In an embodiment, a second ingredient is located on a surface of the elastomeric layer, wherein the second ingredient is selected from the group consisting of a lubricant, a skin penetration enhancing agent, a warming or cooling composition, a skin conditioner, a flavoring, a fragrance, and combinations thereof. Should it be necessary, the second ingredient can be adhesively bonded.

One or more embodiments include condoms having a multiplicity of microcapsules containing an ingredient disposed on both the inner and outer layer of the elastomeric layer of the condom. Other embodiments have encapsulated ingredients disposed on the inner surface or the outer surface of the elastomeric layer of the condom. One or more embodiments of the present invention further include one or more ingredients without encapsulation disposed on one or both surfaces of the elastomeric layer. Other examples of the present invention also include a film on the elastomeric layer, wherein the one or more multiplicities of microcapsules containing one or more ingredients is dispersed in the film. The film can serve to adhere the microcapsules to the elasomeric layer.

In one or more embodiments, the elastomeric layer comprises natural rubber latex, synthetic polyisoprene, guayule, polyurethane, copolymers thereof, or combinations thereof.

In accordance with one aspect of the present invention, the elastomeric layer of some condoms has a thickness in the range of about 0.035 mm to 0.08 mm. Other aspects of the present invention include condoms with elastomeric layers that are contoured to have a larger diameter at defined portions of the condom, or otherwise have a textured surface.

Further embodiments provide that the microcapsules are liposomes having a PDE-5 inhibitor encapsulated therein.

In a detailed aspect, provided are condoms comprising an elastomeric layer and a multiplicity of microcapsules having a PDE-5 inhibitor contained therein, the microcapsules being disposed on a surface of the elastomeric layer such that the microcapsules are substantially exposed, wherein the PDE-5 inhibitor is selected from the group consisting of sildenafil, tadalafil, vardenafil, derivatives thereof, and pharmaceutically acceptable salts thereof. In an embodiment, the PDE-5 inhibitor is present in an amount in the range of about 0.1 to about 100 mg. Another embodiment provides that the range is 3 mg to 10 mg. In another embodiment, the microcapsules containing the PDE-5 inhibitor are lipid vesicles formed from a membrane incorporating a material selected from the group consisting of a phospholipid, a glycolipid, a derivatized lipid, and compatible combinations thereof.

A further aspect provides methods of manufacturing a condom, the methods comprising the steps of: providing a multiplicity of microcapsules having an ingredient therein; forming an elastomeric layer, wherein the elastomeric layer is selected from a group consisting of natural rubber latex, synthetic polyisoprene, guayule, polyurethane and copolymers or combinations thereof; and applying the multiplicity of microcapsules to a surface of the elastomeric layer, such that the microcapsules are substantially exposed. An embodiment provides that the method further comprises forming a film on the elastomeric layer for adhering the multiplicity of microcapsules prior to application of the microcapsules. Another embodiment provides that the providing step further comprises forming liposomes having a PDE-5 inhibitor selected from the group consisting of sidenafil, tadalafil and vardenafil therein. In another embodiment, the method further comprises packaging the condom. In yet another embodiment, the additional step of disposing an ingredient without encapsulation on one or both surfaces of the elastomeric layer is also provided. One or more embodiments also include a step of forming a film on the elastomeric layer prior to application of an encapsulated ingredient, followed by application of an encapsulated ingredient to the layer of film. These embodiments also allow for the encapsulated ingredient to remain exposed or uncovered by any other elastomeric layer after application.

In another aspect, methods for treating sexual dysfunction are provided. The methods comprises providing a condom comprising an elastomeric layer and a multiplicity of microcapsules having a PDE-5 inhibitor contained therein, the microcapsules being disposed on a surface of the elastomeric layer such that the microcapsules are substantially exposed, wherein the PDE-5 inhibitor is selected from the group consisting of sildenafil, tadalafil, vardenafil, derivatives thereof, and pharmaceutically acceptable salts thereof. In a detailed embodiment, the treatment of sexual dysfunction is for males. Other embodiments provide that the treatment is for females. Still further, treatment can be for both males and females.

The term “microbicide,” as used herein, refers to substances active against germs or microbes such as bacteria, viruses and fungi, including those which cause sexually transmitted diseases. Microbicide also refers to a substance having a purpose of reducing the infectivity of microbes and may also be referred to as a bacteriostatic agent, bacteriocidal agent, fungistatic agent, fungicidal agent, viristatic agent and viricidal agent. Such substances may be absorbed into the skin or be retained on the skin surface and should not react adversely with any of the other ingredients, encapsulants and/or films used in some embodiments of the present invention. Examples of microbicides used in some embodiments of the present invention include, without limitation, sodium dodecyl sulfate, nonoxonol-9, quaternary ammonium salts, i.e., benzalkonium chloride and the like, halogenated hydroxy aromatics, chlorhexidine gluconate, triclosan, miconazole, chlortrimazole, as well as pharmacologically acceptable salts thereof. Some of the agents listed above as microbicides, including but not limited to nonoxonol-9 and the like, are also effective as spermicidical agents. One or more embodiments of the present invention utilize other microbicides known in the art.

The term “anti-viral agent,” as used herein, refers to a substance active against viruses. Such substances should be absorbed readily into the skin and should not react adversely with any of the other ingredients, capsules and/or films used in some embodiments of the present invention. One embodiment of the present invention utilizes zinc salts as an anti-viral agent, while other embodiments utilize other anti-viral agents known in the art.

The term “warming lubricant,” as used herein, refers to a substance which generates or creates the feeling of heat to the skin. Examples of suitable warming lubricants are provided in U.S. Patent Application Publication Nos. 2006/0189493 (Chuah) and 2006/0188528 (Chuah). The term “cooling lubricant,” as used herein, refers to a substance which generates or creates the feeling of coolness to the skin. Such substances should be absorbed readily into the skin and should not react adversely with any of the other ingredients, capsules and/or films used in some embodiments of the present invention.

The term “vasodilator,” as used herein, refers to a substance which causes vasodilation, enhances blood circulation to body parts, increases nitrous oxide levels, general heat or create the feeling of heat and/or increase sexual arousal, response or otherwise treat sexual dysfunction. Such substances should be absorbed readily into the skin and should not react adversely with any of the other ingredients, capsules and/or films used in some embodiments of the present invention. Embodiments of the present invention utilize selective inhibitors of phosphodiesterase, for example, cyclic guanosine monophosphate (cGMP) specific phosphodiesterase type 5 (“PDE-5”). PDE-5 inhibitors include, but are not limited to, sildenafil, tadalafil, vardenafil, derivatives, and pharmaceutically acceptable salts thereof. Other suitable vasodilators include prostaglandin compounds, for example prostaglandin E-1, which is known under the pharmaceutical name of alprostadil. Another suitable ingredient is testosterone.

Other embodiments may include compounds which have vasodilatory effects which include niacin, L-arginine, ginger, menthol, nitric oxide, precursors of nitric oxide, i.e., glyceryl trinitrate, organic nitrites, i.e., amyl nitrite, isoamyl nitrite and the like, and variations thereof, while other embodiments utilize other vasodilators known in the art.

In some embodiments, incorporation of a pharmaceutically acceptable vehicle including, but not limited to, lubricious materials, skin penetration enhancement agents, solubility enhancement agents and compatible combinations thereof is envisioned and considered within the scope of the present invention.

While not intending to being bound to a particular theory, it is believed that the localized transdermal transport of materials known to have an inhibitory effect on cyclic guanosine monophosphate (cGMP) specific phosphodiesterase type 5 (PDE-5), results in an increase in the concentration of nitric oxide (NO) in the corpus cavernosum of the penis. This increase in the NO concentration is believed to result in smooth muscle relaxation, i.e., vasodilation, in the corpus cavernosum with a concomitant increased inflow of blood and an erection.

The term “skin conditioner,” as used herein, refers to a substance moisturizing, anti-inflammatory, or other therapeutic qualities. Further, the term “skin conditioner” includes a substance which help prevent or treat skin conditions such as dry skin, loss of skin elasticity and collagen content. Such substances should be absorbed readily into the skin and should not react adversely with any of the other ingredients, capsules and/or films used in some embodiments of the present invention. Embodiments of the present invention utilize skin conditioners such as aloe vera and glycerin, while other embodiments incorporate other skin conditioners known in the art.

In accordance with one aspect of the invention, spermicides, flavorings and fragrances known in the art may be used in one or more embodiments of the present invention. These ingredients are typically retained on the skin surface and should not react adversely with any of the other ingredients, capsules and/or films used in some embodiments of the present invention. The ingredients used in any embodiment of the present invention should be safe for all recommended uses.

The embodiments of the present invention include one or more ingredients which are encapsulated. The terms “encapsulants”, “encapsulated”, “microencapsulated”, “closed capsule” and the like generally refer to a core material being surrounded by another material that serves to preserve or isolate the core material from the environment until a preselected set of conditions occurs. The encapsulants may include materials such as liposomes, gelatins, cellulosics, chemically modified cellulosics, waxes, polymeric resins, fats and the like, as well as combinations of these materials. In some embodiments of the present invention, the encapsulant encompasses vesicles formed from simple or complex lipids, particularly phospholipids, glycolipids, derivatized lipids and other natural or synthetic lipids having cationic, anionic and/or neutral properties. Lipoproteins or lipopolysaccharides can also be incorporated into the membrane of the vesicles formed from these materials as well as membrane stabilizing agents including, but not limited to cholesterol, derivatives of cholesterol, polyethylene glycol, derivatives of polyethylene glycol and the like. Lipid-based vesicles are also referred to as “liposomes”. With liposomes, active materials can be encapsulated, for example, by passive entrapment, active loading, or membrane incorporation.

In some embodiments, such encapsulated ingredients utilize frangible capsules which preselectively rupture, break or dissolve upon the application of moisture, pressure, friction, heat and combinations thereof, that are present during the condom's intended use, for releasing the encapsulated ingredient from the multiplicity of capsules disposed thereon. In some embodiments, encapsulated ingredients having different types of encapsulation may be utilized to provide for particular release profiles and characteristics or ingredients. In other embodiments, the shape and/or wall thickness of the capsules can be varied to allow for timed, staggered or extended release of the ingredients contained within. One or more embodiments include capsules which do not interact or interfere with the ingredients held within or any of the other ingredients, capsules and/or films disposed on the elastomeric layer. In one or more embodiments, a multiplicity of capsules containing encapsulated ingredients is applied to a film on a surface of the elastomeric layer. In such embodiments, the encapsulated material may be situated on the surface or at varied depths within the film, thereby varying the amount of moisture, pressure and/or heat necessary to rupture the capsule and release the contents. Some embodiments of the present invention incorporate films comprised of polyurethane, while other embodiments use other substances known in the art. In accordance with the present invention, the capsules can be of any size, including in the range of about 1 to 1000 micrometers and in the range of about 1 to 1000 nanometers in length. Further advantages of encapsulation of particular ingredients include providing a separation between ingredients which may adversely interact as well as providing improved shelf storage life for ingredients that are subject to deterioration.

In some embodiments, the multiplicity of microcapsules having encapsulated ingredients therein may form discrete and defined layers on the elastomeric layer, while in other embodiments the microcapsules may form intermixed layers upon application to the elastomeric layer. Further, the microcapsules containing encapsulated ingredients and the ingredients without encapsulation may also form discrete and defined layers or form intermixed layers upon application.

In some embodiments, the elastomeric layer is comprised of natural rubber latex, synthetic polyisoprene, guayule, polyurethane, copolymers or combinations thereof, in the general shape of a male or female condom. The term “natural rubber latex” as used in this disclosure encompasses cured elastomeric material sourced from Hevea brasiliensis (the traditional rubber tree), Parthenium argentatum (guayule), sunflower, goldenrod and the like, as well as genetically modified variations of these or other biological sources.

Other embodiments utilize other substances to form the elastomeric layer that are known in the art. In some embodiments, the elastomeric layer is flexible and impermeable.

In accordance with one aspect of the invention, the elastomeric layer can be thin. For example, some embodiments utilize elastomeric layers having a thickness of less than about 0.05 mm. Other embodiments utilize elastomeric layers having a thickness of less than about 0.045 mm (or 0.04 mm, or even 0.035 mm).

Yet other embodiments of the present invention have an elastomeric layer with a textured surface. Some embodiments include structural embellishments, including, but not limited to, ribs, projections, combinations of ribs and projections and the like on at least a portion of the inner and/or outer surface of the elastomeric layer.

Further embodiments of the present invention include a packaging containing the condom, as otherwise described herein. Examples of packaging include, without limitation, wrappers, boxes, tubs having a sealed enclosure and combinations thereof.

One or more embodiments of the present invention include a method for preparing a condom having an encapsulated ingredient including the following steps: forming an elastomeric layer in the shape of a male or female condom; encapsulating an ingredient selected from the group including a microbicide, an anti-viral agent, a warming or cooling lubricant, a vasodilator, a spermicide, a skin conditioner, a flavoring and a fragrance; and disposing the encapsulated ingredient on the elastomeric layer. In one embodiment, the elastomeric layer is made of natural rubber latex, synthetic polyisoprene, guayule, polyurethane and copolymers or combinations thereof. In other embodiments, an ingredient without encapsulation is also disposed on the elastomeric layer.

According to one embodiment of the present invention, the step of disposing an encapsulated ingredient on the formed elastomeric layer includes disposition on the inner and outer layer of the elastomeric layer. In other embodiments, the step of disposing an encapsulated ingredient is limited to the inner or outer surface of the elastomeric layer. This step includes an adhesive that bonds encapsulated ingredient to the elastomeric layer.

A further embodiment also includes disposing a film on one or both surfaces of the elastomeric layer prior to disposing an encapsulated ingredient. In some embodiments the film is a polymer such as polyurethane or any other material known in the art for adhering capsules to latex. The polymer prevents, for example direct contact between a liposome capsule and a latex product.

In other embodiments, the process for manufacturing condoms produces formed elastomeric layer having a defined thickness. In these embodiments, the elastomeric layer can be less than about 0.05 mm thick, while other embodiments have elastomeric layers having a thickness between about 0.035 to about 0.045 mm.

Still other embodiments of the present invention include a formed elastomeric layer having a contoured shape. For example, the open end of the condom has a larger diameter than the remaining length of the formed condom. In other embodiments, the closed end of the condom has a larger diameter than the remaining length of the condom. Yet other embodiments have enlarged diameters at both the closed and opened ends of the condom. In one or more embodiments the enlarged or contoured portion of the condom has a diameter of about 56 mm. In yet other embodiments, the process produces condoms having an elastomeric layer with a textured surface or comprising other structural embellishments.

The condom of the invention may include a lubricious material either as a separate encapsulant or, in some embodiments, as an encapsulated or unencapsulated vehicle for encapsulated ingredients. The lubricious material referred to in the examples may serve as a carrier or vehicle and may be an aqueous based modified cellulosic material, such as a carboxy or hydroxy cellulose, cellulosic derivatives, and the like; polyethylene glycols, polyoxymethylene glycols; a “dry” silicone based material such as polydimethyl siloxanes; an anhydrous material that functions by contact with moisture present on the skin or membranes of the users; and combinations of these materials with other materials known to those skilled in the art of personal lubricants. Further, these materials may include one or more compounds known to enhance the penetration of compounds into human skin or membranes. It is believed that incorporation of compounds such as fatty acids, amine derivatives of fatty acids and lower alkyl sulfoxides into the vehicle may enhance the penetration of active agents into skin.

At least one embodiment of the present invention includes the step of packaging the finished condom. Specifically, in some embodiments, the formed elastomeric layer having an encapsulated ingredient disposed thereon is packaged in a wrapper, box, tub with sealed enclosure or any combination thereof. At least one embodiment of a method of manufacturing a condom of the present invention includes the step of packaging the finished condom. Specifically, in some embodiments, the formed elastomeric layer having at least one ingredient disposed thereon is packaged in a wrapper, box, tub with sealed enclosure, or any combination thereof. In some embodiments, the materials used in forming the package are selected from materials that can form packages that are substantially impermeable to the passage of air and moisture. The packaging materials include, but are not limited to, cellulosic materials, such as paper or paperboard; polymeric films and sheeting; metallic foils; and composite materials formed from two or more of these materials. The packages may be sealed by bonding processes including, but not limited to, adhesive, heat, ultrasonic welding, pressure and combinations of these bonding processes.

Processes that can be useful in making condoms in accordance with various aspects of the present invention are well known to those of ordinary skill in the art.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

### EXAMPLES

A series of prophetic examples of condoms of the invention having one or more ingredients present is shown below. These and other exemplary ingredients may be present in larger or smaller quantities on one or more surfaces of the condom for particular applications and are as follows:

### Example 1A

Sildenafil, optionally mixed with a carrier, is encapsulated in a membrane formed from a material selected from a phospholipid, glycolipid, a derivatized lipid and other natural or synthetic lipids having cationic, anionic and/or neutral properties to form liposomes containing sildenafil. A multiplicity of sildenafil-containing liposomes are applied to one or more surfaces of a condom, which is still tacky after curing. A condom may have between about 0.1 to about 100 mg of the encapsulated sildenafil on the surface so that the encapsulated material is released as the condom is being used. The condom optionally has between about 0.1 to about 5.0 ml of an unencapsulated lubricious material.

### Example 1B

Sildenafil, optionally mixed with a carrier, is encapsulated in a membrane formed from a material selected from a phospholipid, glycolipid, a derivatized lipid and other natural or synthetic lipids having cationic, anionic and/or neutral properties to form liposomes containing sildenafil. A multiplicity of sildenafil-containing liposomes are applied to one or more surfaces of a condom and bonded using polyurethane. A condom may have between about 0.1 to about 100 mg of the encapsulated sildenafil on the surface so that the encapsulated material is released as the condom is being used. The condom optionally has between about 0.1 to about 5.0 ml of an unencapsulated lubricious material.

### Example 2A

Vardenafil, optionally mixed with a carrier, is encapsulated in a membrane formed from a material selected from a phospholipid, glycolipid, a derivatized lipid and other natural or synthetic lipids having cationic, anionic and/or neutral properties to form liposomes contaning vardenafil. A multiplicity of vardenafil containing liposomes are applied to one or more surfaces of a condom, which is still tacky after curing. A condom may have between about 0.1 to about 20 mg of the encapsulated vardenafil on the surface so that the encapsulated material is released as the condom is being used. The condom optionally has between about 0.1 to about 5.0 ml of an unencapsulated lubricious material.

### Example 2B

Vardenafil, optionally mixed with a carrier, is encapsulated in a membrane formed from a material selected from a phospholipid, glycolipid, a derivatized lipid and other natural or synthetic lipids having cationic, anionic and/or neutral properties to form liposomes contaning vardenafil. A multiplicity of vardenafil containing liposomes are applied to one or more surfaces of a condom and bonded using a polyacrylate. A condom may have between about 0.1 to about 20 mg of the encapsulated vardenafil on the surface so that the encapsulated material is released as the condom is being used. The condom optionally has between about 0.1 to about 5.0 ml of an unencapsulated lubricious material.

### Example 3A

Tadalafil, optionally mixed with a carrier, is encapsulated in a membrane formed from a material selected from a phospholipid, glycolipid, a derivatized lipid and other natural or synthetic lipids having cationic, anionic and/or neutral properties to form liposomes containing tadalafil. A multiplicity of tadalafil containing liposomes are applied to one or more surfaces of a condom bonded, which is still tacky after curing. A condom may have between about 0.1 to about 20 mg of the encapsulated tadalafil on the surface so that the encapsulated material is released as the condom is being used. The condom optionally has between about 0.1 to about 5.0 ml of an unencapsulated lubricious material.

### Example 3B

Tadalafil, optionally mixed with a carrier, is encapsulated in a membrane formed from a material selected from a phospholipid, glycolipid, a derivatized lipid and other natural or synthetic lipids having cationic, anionic and/or neutral properties to form liposomes contaning tadalafil. A multiplicity of tadalafil containing liposomes are applied to one or more surfaces of a condom bonded using a solvent based polymer. A condom may have between about 0.1 to about 20 mg of the encapsulated tadalafil on the surface so that the encapsulated material is released as the condom is being used. The condom optionally has between about 0.1 to about 5.0 ml of an unencapsulated lubricious material.

### Example 4A

Glyceryl trinitrate, optionally mixed with a carrier, is encapsulated in a membrane formed from a material selected from a phospholipid, glycolipid, a derivatized lipid and other natural or synthetic lipids having cationic, anionic and/or neutral properties to form liposomes contaning glyceryl trinitrate. A multiplicity of glyceryl trinitrate containing liposomes are applied to one or more surfaces of a condom, which is still tacky after curing. A condom may have between about 0.1 to about 10 mg of the encapsulated glyceryl trinitrate on the surface so that the encapsulated material is released as the condom is being used. The condom optionally has between about 0.1 to about 5.0 ml of an unencapsulated lubricious material.

### Example 4B

Glyceryl trinitrate, optionally mixed with a carrier, is encapsulated in a membrane formed from a material selected from a phospholipid, glycolipid, a derivatized lipid and other natural or synthetic lipids having cationic, anionic and/or neutral properties to form liposomes contaning glyceryl trinitrate. A multiplicity of glyceryl trinitrate containing liposomes are applied to one or more surfaces of a condom using acetone dissolved methyl cellulose. A condom may have between about 0.1 to about 10 mg of the encapsulated glyceryl trinitrate on the surface so that the encapsulated material is released as the condom is being used. The condom optionally has between about 0.1 to about 5.0 ml of an unencapsulated lubricious material.

### Example 5

Sildenafil, optionally mixed with a carrier, is encapsulated. A microbicide is encapsulated. The encapsulated sildenafil and the encapsulated microbicide are applied to one or more surfaces of a condom. A condom may have between about 0.1 to about 100 mg of the encapsulated sildenafil and between about 0.1 to about 0.5 g of the microbicide on the surface. The condom optionally has between about 0.1 to about 5.0 ml of an unencapsulated lubricious material, which may be suitable to spread the microbicide over the skin surface of the users.

### Example 6

Vardenafil, optionally mixed with a carrier, is encapsulated. A microbicide is encapsulated. The encapsulated vardenafil and the encapsulated microbicide are applied to one or more surfaces of a condom. A condom may have between about 0.1 to about 20 mg of the encapsulated vardenafil and between about 0.1 to about 0.5 g of the microbicide. The condom optionally has between about 0.1 to about 5.0 ml of an unencapsulated lubricious material, which may be useful to spread the microbicide over the skin surface of the users.

### Example 7

Tadalafil, optionally mixed with a carrier, is encapsulated. A microbicide is encapsulated. The encapsulated tadalafil and the encapsulated microbicide are applied to one or more surfaces of a condom. A condom may have between about 0.1 to about 20 mg of the encapsulated tadalafil and about 0.1 to about 0.5 g of the microbicide on the surface so that the encapsulated material is exposed to the users' skin surface as the condom is being used. The condom has between about 0.1 to about 5.0 ml of an unencapsulated lubricious material, which may be suitable to spread the microbicide over the skin surface of the users.

### Example 8

Glyceryl trinitrate is mixed with a carrier and encapsulated. A microbicide is encapsulated. The encapsulated glyceryl trinitrate and the encapsulated microbicide are applied to one or more surfaces of a condom. A condom may have between about between about 0.1 to about 10 mg of the encapsulated glyceryl trinitrate and between about 0.1 to about 0.5 g of the encapsulated microbicide. The condom has between about 0.1 to about 5.0 ml of an unencapsulated lubricious material.

### Example 9

Sildenafil, optionally mixed with a carrier, is encapsulated. A fragrance is provided. The encapsulated sildenafil and the fragrance are applied to one or more surfaces of a condom. A condom may have between about between about 0.1 to about 100 mg of the encapsulated sildenafil and between about 0.1 to about 0.5 gm of the fragrance on the surface. The condom optionally has between about 0.1 to about 5.0 ml of an unencapsulated lubricious material.

### Example 10

Vardenafil, optionally mixed with a carrier, is encapsulated. A fragrance is provided. The encapsulated vardenafil and the encapsulated fragrance are applied to one or more surfaces of a condom. A condom may have between about between about 0.1 to about 20 mg of the encapsulated vardenafil and between about 0.1 to about 0.5 g of the fragrance. The condom optionally has between about 0.1 to about 5.0 ml of an unencapsulated lubricious material.

### Example 11

Tadalafil, optionally mixed with a carrier, is encapsulated. A fragrance is provided. The encapsulated tadalafil and the fragrance are applied to one or more surfaces of a condom. A condom may have between about between about 0.1 to about 20 mg of the encapsulated tadalafil and between about 0.1 to about 0.5 g of the fragrance. The condom has between about 0.1 to about 5.0 ml of an unencapsulated lubricious material.

### Example 12

Glyceryl trinitrate, optionally mixed with a carrier, is encapsulated. A fragrance is provided. The encapsulated glyceryl trinitrate and the fragrance are applied to one or more surfaces of a condom. A condom may have between about between about 0.1 to about 10 mg of the encapsulated glyceryl trinitrate and between about 0.1 to about 0.5 g of the fragrance. The condom optionally has between about 0.1 to about 5.0 ml of an unencapsulated lubricious material.

### Example 13

Sildenafil is encapsulated. The encapsulated sildenafil is applied to one or more surfaces of a condom. A condom may have between about between about 0.1 to about 100 mg of the encapsulated sildenafil on the surface so that the encapsulated material is exposed to the users' skin surface as the condom is being used. The condom has between about 0.1 to about 5.0 ml of an unencapsulated lubricious material.

### Example 14

Vardenafil is encapsulated. The encapsulated vardenafil is applied to one or more surfaces of a condom. A condom may have between about between about 0.1 to about 20 mg of the encapsulated vardenafil on the surface so that the encapsulated material is exposed to the users' skin surface as the condom is being used. The condom has between about 0.1 to about 5.0 ml of an unencapsulated lubricious material.

### Example 15

Tadalafil is encapsulated. The encapsulated tadalafil is applied to one or more surfaces of a condom. A condom may have between about between about 0.1 to about 20 mg of the encapsulated tadalafil on the surface so that the encapsulated material is exposed to the users' skin surface as the condom is being used. The condom has between about 0.1 to about 5.0 ml of an unencapsulated lubricious material.

### Example 16

Glyceryl trinitrate is encapsulated. The encapsulated glyceryl trinitrate is applied to one or more surfaces of a condom. A condom may have between about between about 0.1 to about 10 mg of the encapsulated glyceryl trinitrate on the surface so that the encapsulated material is exposed to the users' skin surface as the condom is being used. The condom has between about 0.1 to about 5.0 ml of an unencapsulated lubricious material.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A condom comprising: an elastomeric layer and a multiplicity of microcapsules having an ingredient therein, the microcapsules being disposed on a surface of the elastomeric layer such that the microcapsules are substantially exposed.

2. The condom of claim 1, wherein the microcapsules are bonded to the elastomeric layer.

3. The condom of claim 1, further comprising a barrier layer between the elastomeric layer and the microcapsules.

4. The condom of claim 3, wherein the barrier layer comprises an ingredient selected from the group consisting of nitrile, polyurethane, and polyacetal urea.

5. The condom of claim 1, wherein the ingredient in the microcapsules is selected from the group consisting of a microbicide, an anti-viral agent, a warming lubricant, a cooling lubricant, a vasodilator, a spermicide, and compatible combinations thereof.

6. The condom of claim 1, wherein the ingredient in the microcapsules is a PDE-5 inhibitor.

7. The condom of claim 6, wherein the PDE-5 inhibitor is a compound selected from the group consisting of sildenafil, tadalafil, vardenafil, derivatives thereof, and pharmaceutically acceptable salts thereof.

8. The condom of claim 1, wherein a second ingredient is located on a surface of the elastomeric layer, wherein the second ingredient is selected from the group consisting of a lubricant, a skin penetration enhancing agent, a warming composition, a cooling lubricant, a skin conditioner, a flavoring, a fragrance, and combinations thereof.

9. The condom of claim 1, wherein the microcapsules are disposed on an inner surface of the elastomeric layer.

10. The condom of claim 1, wherein the elastomeric layer comprises natural rubber latex, synthetic polyisoprene, guayule, polyurethane, copolymers thereof, or combinations thereof.

11. The condom of claim 1, further comprising a film on the elastomeric layer, the microcapsules being adhered to the elastomeric layer by the film.

12. The condom of claim 1 wherein the microcapsules are liposomes having a PDE-5 inhibitor encapsulated therein.

13. A condom comprising an elastomeric layer and a multiplicity of microcapsules having a PDE-5 inhibitor contained therein, the microcapsules being disposed and bonded on a surface of the elastomeric layer such that the microcapsules are substantially exposed, wherein the PDE-5 inhibitor is selected from the group consisting of sildenafil, tadalafil, vardenafil, derivatives thereof, and pharmaceutically acceptable salts thereof.

14. The condom of claim 13, wherein the PDE-5 inhibitor comprises sildenafil, derivatives thereof, or pharmaceutically acceptable salts thereof being present in an amount in the range of about 0.1 to about 100 mg.

15. The condom of claim 13, wherein the microcapsules containing the PDE-5 inhibitor are lipid vesicles formed from a membrane incorporating a material selected from the group consisting of a phospholipid, a glycolipid, a derivatized lipid, and compatible combinations thereof.

16. The condom of claim 13, wherein the microcapsules are bonded by an adhesive comprising an ingredient selected from the group consisting of nitrile, polyurethane, and combinations thereof.

17. A method for manufacturing a condom, the method comprising the steps of:
providing a multiplicity of microcapsules having an ingredient therein;
forming an elastomeric layer, wherein the elastomeric layer is selected from a group consisting of natural rubber latex, synthetic polyisoprene, guayule, polyurethane and copolymers or combinations thereof; and
applying the multiplicity of microcapsules to a surface of the elastomeric layer, such that the microcapsules are substantially exposed.

18. The method of claim 17, further comprising adhering the multiplicity of microcapsules to the surface.

19. The method of claim 17, wherein the providing step further comprises forming liposomes having a PDE-5 inhibitor selected from the group consisting of sidenafil, tadalafil and vardenafil therein.

20. A method for treating sexual dysfunction, the method comprising providing a condom comprising an elastomeric layer and a multiplicity of microcapsules having a PDE-5 inhibitor contained therein, the microcapsules being disposed on a surface of the elastomeric layer such that the microcapsules are substantially exposed, wherein the PDE-5 inhibitor is selected from the group consisting of sildenafil, tadalafil, vardenafil, derivatives thereof, and pharmaceutically acceptable salts thereof.
